(19) United States
(12) Patent Application Publication
(10) Pub. No.: US 2025/0000600 A1
(43) Pub. Date: Jan. 2, 2025

(54) GLOVE COMPRISING COATING

(71) Applicant: SHANDONG INTCO MEDICAL PRODUCTS CO., LTD, Qingzhou (CN)

(72) Inventors: Fangyi LIU, Qingzhou (CN); Ruijun LI, Qingzhou (CN); Mingwei SUN, Qingzhou (CN); Enjie HE, Qingzhou (CN); Min ZHAO, Huaibei (CN); Qiuying DONG, Qingzhou (CN)

(21) Appl. No.: 18/361,506

(22) Filed: Jul. 28, 2023

(30) Foreign Application Priority Data
Jun. 30, 2023 (CN) 2023107909620

(51) Int. Cl. A61B 42/10; A61L 31/10

(52) U.S. Cl. CPC A61B 42/10; A61L 31/10

(57) ABSTRACT

The present disclosure relates to the technical field of glove processing, and in particular to a glove comprising a coating and a preparation method thereof. The glove has an inner surface for contact with skin of a subject's hand, the inner surface being provided with a coating comprising hyaluronic acid or its salt thereof, and glycerin. The glove comprising the coating, provided by the present disclosure, is effective in increasing the skin hydration of a wearer's hand; while the coating has a simple preparation process with raw materials readily available, which is suitable for industrial production.

### CROSS REFERENCE TO RELATED PATENT APPLICATION

This application claims the priority of the Chinese patent application No. 202310790962.0, filed on Jun. 30, 2023, which is incorporated herein by reference in its entirety.

### FIELD

The present disclosure relates to the technical field of glove processing, and in particular to a glove comprising a coating and a preparation method thereof.

### BACKGROUND

Disposable gloves are common disposable isolation protective gloves, which are widely used in medical examination, medical care, laboratory, electronic product processing, food processing, fast food service, and other fields. With the continuous development of the world economy and the continuous change in people's consumption concept, people put forward more demands for the function of gloves.

Wearing gloves (such as nitrile gloves) for a long time may cause discomfort to the wearer, especially when the wearer wears gloves for a long period of time, the skin of the hands is liable to be dry and astringent, and there are individuals with allergies and redness. Chinese patent CN202504246U discloses a glove covered with an aloe coating, which has a moisturizing effect on the skin; however, during the preparation of the glove comprising the coating, a sufficient volume of a coating solution needs to be prepared, and the coating solution will be wasted during use, the cost of selecting aloe is relatively high. Chinese patent CN104621798A discloses a green aloe skin-care nitrile glove and the production method thereof; a nitrile glove with skin-care properties is prepared by adding aloe powder in the process of formulating nitrile latex, but there is also a problem of high cost.

### SUMMARY

In one aspect, the present disclosure provides a glove comprising a coating; the glove has an inner surface for contact with skin of a subject's hand, the inner surface being provided with a coating comprising hyaluronic acid or its salt thereof, and glycerin.

In some embodiments, the coating on the inner surface of the glove increases skin hydration of the subject's hand upon contact therewith.

In some embodiments, the coating comprises, by mass parts, 0.06 to 0.29 parts of hyaluronic acid or its salt thereof, and 6 to 29 parts of glycerin.

In some embodiments, the coating comprises, by mass parts, 0.1 to 0.2 parts of hyaluronic acid or its salt thereof, and 10 to 20 parts of glycerin.

In some embodiments, the coating comprises, by mass parts, 0.11 to 0.18 parts of hyaluronic acid or its salt thereof, and 11 to 18 parts of glycerin.

In some embodiments, the coating comprises, by mass parts, 0.12 to 0.17 parts of hyaluronic acid or its salt thereof, and 12 to 17 parts of glycerin.

In some embodiments, the coating comprises, by mass parts, 0.13 to 0.17 parts of hyaluronic acid or its salt thereof, and 13 to 17 parts of glycerin.

In some embodiments, the coating comprises, by mass parts, 0.14 to 0.17 parts of hyaluronic acid or its salt thereof, and 13 to 16 parts of glycerin.

In some embodiments, the coating comprises, by mass parts, 0.14 to 0.16 parts of hyaluronic acid or its salt thereof, and 14 to 16 parts of glycerin.

In some embodiments, the coating comprises, by mass parts, 0.15 to 0.16 parts of hyaluronic acid or its salt thereof, and 14 to 15 parts of glycerin.

In some embodiments, the coating comprises, by mass parts, 0.16 parts of hyaluronic acid or its salt thereof, and 14 parts of glycerin.

In some embodiments, the coating further comprises one of butylene glycol, allantoin, or propylene glycol.

In some embodiments, 1 to 5 parts by mass of butylene glycol, allantoin, or propylene glycol are provided.

In some embodiments, 3 to 4 parts by mass of butylene glycol, allantoin, or propylene glycol are provided.

In some embodiments, 2 parts by mass of butylene glycol, allantoin, or propylene glycol are provided.

In some embodiments, the salt of hyaluronic acid is sodium hyaluronate, potassium hyaluronate, or calcium hyaluronate.

In some embodiments, the salt of hyaluronic acid is sodium hyaluronate.

In another aspect, the present disclosure further provides a coating solution comprising hyaluronic acid or its salt thereof, and glycerin.

In some embodiments, the hyaluronic acid or its salt thereof is 0.06 to 0.29 parts by mass and the glycerol is 6 to 29 parts by mass.

In some embodiments, the coating comprises, by mass parts, 0.1 to 0.2 parts of hyaluronic acid or its salt thereof, and 10 to 20 parts of glycerin.

In some embodiments, the coating comprises, by mass parts, 0.11 to 0.18 parts of hyaluronic acid or its salt thereof, and 11 to 18 parts of glycerin.

In some embodiments, the coating comprises, by mass parts, 0.12 to 0.17 parts of hyaluronic acid or its salt thereof, and 12 to 17 parts of glycerin.

In some embodiments, the coating comprises, by mass parts, 0.13 to 0.17 parts of hyaluronic acid or its salt thereof, and 13 to 17 parts of glycerin.

In some embodiments, the coating comprises, by mass parts, 0.14 to 0.17 parts of hyaluronic acid or its salt thereof, and 13 to 16 parts of glycerin.

In some embodiments, the coating comprises, by mass parts, 0.14 to 0.16 parts of hyaluronic acid or its salt thereof, and 14 to 16 parts of glycerin.

In some embodiments, the coating comprises, by mass parts, 0.15 to 0.16 parts of hyaluronic acid or its salt thereof, and 14 to 15 parts of glycerin.

In some embodiments, the coating comprises, by mass parts, 0.16 parts of hyaluronic acid or its salt thereof, and 14 parts of glycerin.

In some embodiments, the coating further comprises one of butylene glycol, allantoin, or propylene glycol.

In some embodiments, 1 to 5 parts by mass of butylene glycol, allantoin, or propylene glycol are provided.

In some embodiments, 3 to 4 parts by mass of butylene glycol, allantoin, or propylene glycol are provided.

In some embodiments, 2 parts by mass of butylene glycol, allantoin, or propylene glycol are provided.

In some embodiments, the coating solution further comprises water.

In some embodiments, 140 to 175 parts by mass of water are provided.

In some embodiments, 150 to 170 parts by mass of water are provided.

In some embodiments, the salt of hyaluronic acid is sodium hyaluronate, potassium hyaluronate, or calcium hyaluronate.

In some embodiments, the salt of hyaluronic acid is sodium hyaluronate.

In another aspect, the present disclosure further provides a preparation method of a glove, comprising: providing a glove having an inner surface for contact with skin of a subject's hand, wherein the inner surface is provided with a coating comprising hyaluronic acid or its salt thereof, and glycerin.

In another aspect, the present disclosure further provides a preparation method of a glove, comprising the following steps:
S1, formulation of the coating solution: stirring and mixing components to obtain the coating solution; and S2, impregnating a glove in the coating solution prepared in step S1 before removing for drying, to obtain the glove.

In another aspect, the present disclosure further provides a glove having an inner surface for contact with skin of a subject's hand, the glove being prepared by impregnating the inner surface in the above coating solution before removing for drying.

In some embodiments, the glove is a nitrile glove.

In some embodiments, the glove may also be prepared by the following steps:
(1) formulation of the above coating solution: stirring and mixing components to obtain the coating solution;
(2) heating a glove model to 50 to 70° C. before impregnating into a coagulant solution for 1 to 5 seconds, and removing for drying;
(3) impregnating the glove model acquired in step (2) into nitrile latex excipients for 1 to 5 seconds before removing for drying, vulcanization, chlorine washing, and leaching to obtain the nitrile glove, and attaching the nitrile glove to a ceramic hand mold; and
(4) impregnating the glove attached to the ceramic hand mold acquired in step (3) into the coating solution formulated in step (1) before removing for baking and demolding to obtain the glove.

In some embodiments, in step (2), the coagulant solution comprises a soluble salt, a mold release agent, and water.

In some embodiments, the soluble salt is selected from one or more of calcium nitrate, calcium chloride, magnesium nitrate, magnesium chloride, zinc nitrate, and zinc chloride.

In some embodiments, the mold release agent is selected from calcium stearate.

In some embodiments, a mass ratio of the soluble salt, mold release agent, and water is 6 to 12:0.5 to 1.5:70 to 90.

In some embodiments, in step (3), the nitrile latex excipients comprise the following raw material components in mass parts: 5 to 10 parts of zinc oxide powder, 3 to 8 parts of sulfur powder, 0.5 to 3 parts of accelerator BZ powder, 1 to 5 parts of titanium dioxide powder, 0.5 to 3 parts of antioxidant powder, 0.1 to 1.5 parts of dispersant powder, and 130 to 150 parts of water.

In some embodiments, the nitrile latex excipients are prepared by the following method: adding the raw material components of the nitrile latex excipients into a dispersion stirring tank before adding water to disperse and stir for 10 to 20 hours, and performing cyclic grinding on the acquired dispersion in a sand mill for 4 to 8 hours until the particle size of the powder in the dispersion is less than or equal to 5 μm, to obtain the nitrile latex excipients.

In some embodiments, the nitrile latex excipients have a solid content of 45 to 55%.

In some embodiments, the dispersant is NNO.

In some embodiments, the antioxidant is KY-616.

In some embodiments, the particle size of the raw material components of the nitrile latex are less than or equal to 5 μm.

In some embodiments, the rubber-based rubber selected for the present disclosure is nitrile rubber.

In some embodiments, in step (3), the vulcanization temperature is 105 to 135° C.; and the vulcanization time is 0.3 to 0.6 hours.

In some embodiments, in step (4), the impregnation is constant temperature uniform impregnation; the impregnation temperature is 50 to 70° C.; and the impregnation speed is 5 cm/s.

### DETAILED DESCRIPTION

Unless defined otherwise, all technical and scientific terms used herein have the same meaning as commonly understood by one of ordinary skill in the art to which this disclosure belongs.

As used herein, the term “skin hydration” refers to the moisture content of stratum corneum of skin.

As used herein, the term “the change in skin hydration” refers to the change in the moisture content of stratum corneum of skin (%) before and after the subject wears the glove, and the calculation formula is ((skin hydration after wearing-skin hydration before wearing)/skin hydration before wearing)*100%.

The technical solutions of the present disclosure are further illustrated below by the specific embodiments, which are not intended to limit the scope of protection of the present disclosure. Certain insubstantial modifications and adaptations made by others according to the concepts of the present disclosure remain within the scope of protection of the present disclosure.

Sodium hyaluronate is purchased from Dongying First Biochem Industrial Co., Ltd., Art. No. moistuHA®1% sodium hyaluronate aqueous solution (1% mass fraction of sodium hyaluronate in aqueous solution).

Glycerin is purchased from Wujiang Huitong Chemical Co., LTD., Art. No. HT-045.

Allantoin is purchased from Qiansiguang (Guangzhou) Chemical Co., LTD., Art. No. 126464163164.

Aloe extract is purchased from Xi'an TK Bio-technology Co., Ltd., Art. No. TKLH20221017.

Butylene glycol is purchased from Jinan Xinchaorui Chemical Co., Ltd., Art. No. 021.

Propylene glycol is purchased from Dow Chemical Thailand Co., Ltd., Art. No. 0002.

### Example 1 Preparation of Glove Coatings

### (1) Preparation of a Glove Body

At S1, nitrile latex excipients were prepared, where 5 parts of zinc oxide powder, 8 parts of sulfur powder, 3 parts of accelerator BZ powder, 5 parts of titanium dioxide powder, 3 parts of antioxidant powder, 1.5 parts of dispersant powder, and 200 parts of water were added into a dispersion stirring tank and dispersed and stirred for 10 hours to obtain a dispersion. The dispersion was performed with cyclic grinding in a sand mill for 4 hours until the particle size of the powder in the dispersion is less than or equal to 5 μm, to obtain the nitrile latex excipients. The nitrile latex excipients had a solid content of about 45%.

At S2, the glove model was heated to 50 to 70° C. before being impregnated into a coagulant solution for 5 seconds, and removed for drying. The coagulant solution was a calcium nitrate solution, the mold release agent was calcium stearate, and the mass ratio of the coagulant solution, the mold release agent, and water was 6:0.5:70.

At S3, the glove model acquired in step S2 was impregnated into the nitrile latex excipients for 5 seconds before being removed for drying, vulcanization, chlorine washing, and leaching to obtain a nitrile glove.

### (2) Preparation of a Coating Solution

The raw materials of the coating solution shown in Table 1 were mixed before being thoroughly stirred to uniformly disperse.

### (3) Preparation of a Glove Comprising a Coating

The nitrile glove was attached to the ceramic hand mold and impregnated into the coating solution formulated in step (2) for 5 seconds before being removed for baking and demolding to prepare the glove.

### Example 2 Wearing Experiments

The experiment was carried out using the glove comprising the coating prepared in Example 1, with 10 subjects in each group. Before the experiment, the subjects wiped the hands with a paper towel to reduce the effect of sweat secretion; skin hydration on the back of hands before wearing the gloves was tested using a skin tester (IMATE series, available from Shenzhen Fuhengtong Technology Co., Ltd.); the gloves were taken off after wearing for 40 minutes, the sweat that may appear on the skin surface of the hands was gently wiped with a paper towel; skin hydration on the back of hands after wearing the gloves was tested using a skin tester (IMATE series, available from Shenzhen Fuhengtong Technology Co., Ltd.); and finally, the change in skin hydration before and after wearing (%) was calculated. The test results were shown in Table 1. Change in skin hydration before and after wearing %=((skin hydration after wearing-skin hydration before wearing)/skin hydration before wearing)*100%.

TABLE 1
Coating component composition and wearing experiment results in different groups.

| | Coating solution composition | | | | Wearing experiments of the | |
|---|---|---|---|---|---|---|
| | | | | | prepared glove comprising the | |
| | | | Other | | coating and cost calculation of | |
| | | | components | | the coating solution | |
| | Main components | | (mass parts | Water | Change in skin | Coating |
| | (mass parts of 30) | | of 2) | Mass | hydration % | solution |
| | Component A | Component B | Component | parts of | (mean ± | formulation |
| | and mass parts | and mass parts | C and mass | water | standard | cost |
| Group | (parts) | (parts) | parts (parts) | (parts) | deviation) | (yuan/100 kg) |
| A1 | Sodium hyaluronate | Glycerin/14 | Butylene | make | 14.5 ± 2.17 | 444.44 |
| | solution/16 (equivalent | | glycol/2 | up to | | |
| | to sodium hyaluronate | | | 180 | | |
| | of 0.16) | | | | | |
| A2 | Sodium hyaluronate | Glycerin/14 | Propylene | make | 13.76 ± 2.09 | 444.44 |
| | solution/16 (equivalent | | glycol/2 | up to | | |
| | to sodium hyaluronate | | | 180 | | |
| | of 0.16) | | | | | |
| A3 | Sodium hyaluronate | Allantoin/14 | Butylene | make | 9 ± 2.05 | 677.78 |
| | solution/16 (equivalent | | glycol/2 | up to | | |
| | to sodium hyaluronate | | | 180 | | |
| | of 0.16) | | | | | |
| A4 | Allantoin/16 | Glycerin/14 | Butylene | make | 7 ± 2.00 | 622.22 |
| | | | glycol/2 | up to | | |
| | | | | 180 | | |
| A5 | Aloe/16 | Glycerin/14 | Butylene | make | 8.7 ± 2.56 | 622.22 |
| | | | glycol/2 | up to | | |
| | | | | 180 | | |
| A6 | Sodium hyaluronate | Aloe/14 | Allantoin/2 | make | 10.8 ± 2.44 | 711.11 |
| | solution/16 (equivalent | | | up to | | |
| | to sodium hyaluronate | | | 180 | | |
| | of 0.16) | | | | | |
| A7 | Sodium hyaluronate | Aloe/14 | Butylene | make | 12.6 ± 2.37 | 677.78 |
| | solution/16 (equivalent | | glycol/2 | up to | | |
| | to sodium hyaluronate | | | 180 | | |
| | of 0.16) | | | | | |

Note: The sodium hyaluronate solution in component A in Table 1 was an aqueous solution with a mass fraction of sodium hyaluronate of 100. The cost calculation was based on the current market reference price. The average price of an aqueous solution of sodium hyaluronate with a mass fraction of 1% was 25 to 30 yuan/kg; the average price of aloe extract as a cosmetic raw material was 35 to 50 yuan/kg; the average price of allantoin as a cosmetic was 40 to 50 yuan/kg; the average price of butylene glycol was about 10 to 20 yuan/kg; the average price of glycerin was about 10 to 20 yuan; and the average price of propylene glycol was about 10 to 20 yuan/kg.

The glove prepared by applying the coating was examined for the change in skin hydration on the back of the hands of the wearer after a period of wearing. The results showed that the glove comprising the coating of each group increased the skin hydration of the wearer after removing the gloves, and no redness was found in any of the experiment subjects. Compared with the other groups, the skin of the hands of the subjects in groups A1 and A2 was clearer without a greasy feeling after removing the gloves.

It was found that as the formulation composition of the glove coating, the compound groups of sodium hyaluronate+glycerin (groups A1 and A2) had the best effect, which was significantly better than the sodium hyaluronate+allantoin group (group A3) (P<0.05), the aloe+glycerin group (group A5) (P<0.05), or the allantoin+glycerin group (group A4) (P<0.05), and slightly better than the effect of the sodium hyaluronate+aloe group (groups A6 and A7). The cost was an important consideration because, for the preparation process for the glove using the coating solution, a sufficient volume of coating solution needed to be formulated, and there was some waste of coating solution. Glycerin, as a low-cost raw material, enabled the skin-care effect of gloves in the compound group of sodium hyaluronate to be exerted well, which replaced the aloe coating as a glove comprising a coating with a lower manufacturing cost as well as a better effect. The glove of group A1 had the best skin-care effect and the glove of group A2 had comparatively good skin-care effect, while the cost of group A1 and group A2 was at the lowest level, which was an ideal glove coating composition.

### Example 3 Preparation of Glove Coating

(1) preparation of a glove body: The preparation method was the same as that in Example 1.
(2) preparation of a coating solution: The raw materials of the coating solution shown in Table 2 were mixed before being thoroughly stirred to uniformly disperse.
(3) preparation of a glove comprising a coating: The nitrile glove was attached to the ceramic hand mold and impregnated into the coating solution formulated in step (2) for 5 seconds before being removed for baking and demolding to prepare the glove.

### Example 4 Wearing Experiments

The experiment was carried out using the glove comprising the coating prepared in Example 3, with 10 subjects in each group. Before the experiment, the subjects wiped the hands with a paper towel to reduce the effect of sweat secretion; skin hydration on the back of the hands before wearing the gloves was tested using a skin tester (IMATE series, available from Shenzhen Fuhengtong Technology Co., Ltd.); the gloves were taken off after wearing for 40 minutes, the sweat that may appear on the skin surface of the hands was gently wiped with a paper towel; skin hydration on the back of hands after wearing the gloves was tested using a skin tester (IMATE series, available from Shenzhen Fuhengtong Technology Co., Ltd.); and finally, the change in skin hydration before and after wearing (0%) was calculated. The test results were shown in Table 2 below. Change in skin hydration before and after wearing %=((skin hydration after wearing-skin hydration before wearing)/skin hydration before wearing)*100%.

TABLE 2
Coating component composition and wearing experiment results in different groups.

| | | | | | Wearing experiments of the | |
|---|---|---|---|---|---|---|
| | | | | | prepared glove comprising the | |
| | | | | | coating and cost calculation of | |
| | | | | | the coating solution | |
| | Coating solution composition | | | | | Cost |
| | | | Other | Water | Change in skin | Calculation |
| | Main components | | components | Mass | hydration % | of Coating |
| | Component | Component | Component | parts of | (mean ± | Solution |
| | A and mass | B and mass | C and mass | water | standard | formulation |
| Group | parts (parts) | parts (parts) | parts (parts) | (parts) | deviation) | (yuan/100 kg) |
| B8 | Sodium hyaluronate | Glycerin/10 | Butylene | make | 9.5 ± 2.17 | 333.33 |
| | solution/12(equivalent | | glycol/2 | up to | | |
| | to sodium hyaluronate | | | 180 | | |
| | of 0.12) | | | | | |
| B9 | Sodium hyaluronate | Glycerin/10 | Propylene | make | 6.98 ± 2.05 | 333.33 |
| | solution/12 (equivalent | | glycol/2 | up to | | |
| | to sodium hyaluronate | | | 180 | | |
| | of 0.12) | | | | | |
| B10 | Sodium hyaluronate | Aloe/10 | Allantoin/2 | make | 6.3 ± 1.49 | 506.67 |
| | solution/12 (equivalent | | | up to | | |
| | to sodium hyaluronate | | | 180 | | |
| | of 0.12) | | | | | |
| C11 | Sodium hyaluronate | Glycerin/12 | Butylene | make | 10 ± 1.94 | 388.89 |
| | solution/14 (equivalent | | glycol/2 | up to | | |
| | to sodium hyaluronate | | | 180 | | |
| | of 0.14) | | | | | |
| C12 | Sodium hyaluronate | Glycerin/12 | Propylene | make | 9.78 ± 2.09 | 388.89 |
| | solution/14 (equivalent | | glycol/2 | up to | | |
| | to sodium hyaluronate | | | 180 | | |
| | of 0.14) | | | | | |
| C13 | Sodium hyaluronate | Aloe/12 | Allantoin/2 | make | 7.4 ± 1.71 | 622.22 |
| | solution/14 (equivalent | | | up to | | |
| | to sodium hyaluronate | | | 180 | | |
| | of 0.14) | | | | | |
| D14 | Sodium hyaluronate | Glycerin/12 | Butylene | make | 11.5 ± 1.84 | 422.22 |
| | solution/16 (equivalent | | glycol/2 | up to | | |
| | to sodium hyaluronate | | | 180 | | |
| | of 0.16) | | | | | |
| D15 | Sodium hyaluronate | Glycerin/12 | Propylene | make | 10.2 ± 2.98 | 422.22 |
| | solution/16 (equivalent | | glycol/2 | up to | | |
| | to sodium hyaluronate | | | 180 | | |
| | of 0.16) | | | | | |
| D16 | Sodium hyaluronate | Aloe/12 | Allantoin/2 | make | 9.2 ± 1.14 | 655.56 |
| | solution/16 (equivalent | | | up to | | |
| | to sodium hyaluronate | | | 180 | | |
| | of 0.16) | | | | | |
| E17 | Sodium hyaluronate | Glycerin/14 | Butylene | make | 14.5 ± 2.17 | 444.44 |
| | solution/16 (equivalent | | glycol/2 | up to | | |
| | to sodium hyaluronate | | | 180 | | |
| | of 0.16) | | | | | |
| E18 | Sodium hyaluronate | Glycerin/14 | Propylene | make | 13.76 ± 2.09 | 444.44 |
| | solution/16 (equivalent | | glycol/2 | up to | | |
| | to sodium hyaluronate | | | 180 | | |
| | of 0.16) | | | | | |
| E19 | Sodium hyaluronate | Aloe/14 | Allantoin/2 | make | 10.8 ± 2.44 | 711.11 |
| | solution/16 (equivalent | | | up to | | |
| | to sodium hyaluronate | | | 180 | | |
| | of 0.16) | | | | | |
| F20 | Sodium hyaluronate | Glycerin/18 | Butylene | make | 10.1 ± 1.66 | 405.56 |
| | solution/11 (equivalent | | glycol/2 | up to | | |
| | to sodium hyaluronate | | | 180 | | |
| | of 0.11) | | | | | |
| F21 | Sodium hyaluronate | Glycerin/18 | Propylene | make | 7.44 ± 2.03 | 405.56 |
| | solution/11 (equivalent | | glycol/2 | up to | | |
| | to sodium hyaluronate | | | 180 | | |
| | of 0.11) | | | | | |
| F22 | Sodium hyaluronate | Aloe/18 | Allantoin/2 | make | 5.4 ± 2.01 | 738.89 |
| | solution/11 (equivalent | | | up to | | |
| | to sodium hyaluronate | | | 180 | | |
| | of 0.11) | | | | | |

Note: The sodium hyaluronate solution in component A in Table 2 was an aqueous solution with a mass fraction of sodium hyaluronate of 100. The cost calculation was based on the current market reference price. The average price of an aqueous solution of sodium hyaluronate with a mass fraction of 1% is 25 to 30 yuan/kg; the average price of aloe extract as a cosmetic raw material was 35 to 50 yuan/kg; the average price of allantoin as a cosmetic was 40 to 50 yuan/kg; the average price of butylene glycol was about 10 to 20 yuan/kg; the average price of glycerin was about 10 to 20 yuan; and the average price of propylene glycol was about 10 to 20 yuan/kg.

The glove prepared by applying the coating was examined for the change in skin hydration on the back of the hands of the wearer after a period of wearing. The results showed that the glove comprising the coating of each group increased the skin hydration of the wearer after removing the gloves, and no redness was found in any of the experiment subjects. Compared with the other groups, the skin of the hands of the subjects in groups B8, B9, C11, C12, D14, D15, E17, E18, F20 and F21 was clearer without a greasy feeling after removing the gloves.

It was found that as the formulation composition of the glove coating, the compound group of sodium hyaluronate+glycerin+butylene glycol (group B8, group C11, group D14, group E17, and group F20) had a better effect, which was embodied in that group B8 was significantly better than the sodium hyaluronate+aloe+allantoin group (group B10) (P<0.05); group C11 was significantly better than the sodium hyaluronate+aloe+allantoin group (group C13) (P<0.05); group D14 was significantly better than the sodium hyaluronate+aloe+allantoin group (group D16) (P<0.05); group E17 was significantly better than the sodium hyaluronate+aloe+allantoin group (group E19) (P<0.05); and group F20 was significantly better than the sodium hyaluronate+aloe+allantoin group (group F22) (P<0.05).

Similarly, the cost was an important consideration because, for the preparation process for the glove using the coating solution, a sufficient volume of coating solution needed to be formulated, and there was some waste of coating solution. Glycerin, butylene glycol or propylene glycol, as low-cost raw materials, enabled the skin-care effect of gloves in the compound group of sodium hyaluronate to be exerted well, while the cost was at a low level, which was an ideal glove coating composition.

Furthermore, as the formulation composition of the glove coating, the “sodium hyaluronate+glycerol+propylene glycol” group, although not as effective as the “sodium hyaluronate+glycerol+butylene glycol” group as a whole, was significantly superior to the other groups. For example: group C12 was significantly better than the sodium hyaluronate+aloe+allantoin group (group C13) (P<0.05), group D15 was significantly better than the sodium hyaluronate+aloe+allantoin group (group D16) (P<0.05), group E18 was significantly better than the sodium hyaluronate+aloe+allantoin group (group E19) (P<0.05), group F21 was significantly better than the sodium hyaluronate+aloe+allantoin group (group F22) (P<0.05). Although group B9 was not significantly better than the sodium hyaluronate+aloe+allantoin group (group B10) (P>0.05), group B9 had a much lower cost of preparation than group B10 with a slightly better effect than group B10. Therefore, glycerin or propylene glycol, as low-cost raw materials, enabled the skin-care effect of gloves in the compound group of sodium hyaluronate to be exerted well, while the cost was at a comparatively low level, which was also an ideal glove coating composition.

Compared with the groups of sodium hyaluronate+glycerin+propylene glycol, the groups of sodium hyaluronate+glycerin+butylene glycol had a more outstanding advantage, which had significantly better moisturizing effect in a wider range of sodium hyaluronate-to-glycerin ratio. As for the groups of sodium hyaluronate+glycerin+propylene glycol, to further improve moisturizing effect, the sodium hyaluronate-to-glycerin ratio needed to be more strictly compounded. Considering both moisturizing effect and cost, both groups were significantly better than the others.

### Example 5 Glove Coating Preparation

(1) preparation of a glove body: The preparation method was the same as that in Example 1.
(2) preparation of a coating solution: The raw materials of the coating solution shown in Table 3 were mixed before being thoroughly stirred to uniformly disperse.
(3) preparation of a glove comprising a coating: The nitrile glove was attached to the ceramic hand mold and impregnated into the coating solution formulated in step (2) for 5 seconds before being removed for baking and demolding to prepare the glove.

### Example 6 Wearing Experiments

The experiment was carried out using the glove comprising the coating prepared in Example 5, with 10 subjects in each group. Before the experiment, the subjects wiped the hands with a paper towel to reduce the effect of sweat secretion; skin hydration on the back of hands before wearing the gloves was tested using a skin tester (IMATE series, available from Shenzhen Fuhengtong Technology Co., Ltd.); the gloves were taken off after wearing for 40 minutes, the sweat that may appear on the skin surface of the hands was gently wiped with a paper towel; skin hydration on the back of hands after wearing the gloves was tested using a skin tester (IMATE series, available from Shenzhen Fuhengtong Technology Co., Ltd.); and finally, the change in skin hydration before and after wearing ( ) was calculated. The test results were shown in Table 3 below. Change in skin hydration before and after wearing %=((skin hydration after wearing-skin hydration before wearing)/skin hydration before wearing)*100%.

TABLE 3
Coating component composition and wearing experiment results in different groups.

| | | | | | Wearing |
|---|---|---|---|---|---|
| | | | | | experiments of |
| | | | | | the prepared |
| | | | | | glove comprising the |
| | | | | | coating |
| | Coating solution composition | | | | Change in skin |
| | Main components | | Other | | hydration % |
| | | Component | components | Water | (mean ± |
| | Component A and | B and mass | Component C and | Mass parts of | standard |
| Group | mass parts (parts) | parts (parts) | mass parts (parts) | water (parts) | deviation) |
| G1 | Sodium hyaluronate | Glycerin/ | Butylene glycol/ | make up to | 9.5 ± 2.17 |
| | solution/12 | 10 | 2 | 180 | |
| | (equivalent to | | | | |
| | sodium hyaluronate | | | | |
| | of 0.12) | | | | |
| G2 | Sodium hyaluronate | Glycerin/ | Butylene glycol/ | make up to | 7.1 ± 2.39 |
| | solution/30 | 10 | 2 | 180 | |
| | (equivalent to | | | | |
| | sodium hyaluronate | | | | |
| | of 0.3) | | | | |
| G3 | Sodium hyaluronate | Glycerin/ | Butylene glycol/ | make up to | 5.9 ± 1.22 |
| | solution/5 | 10 | 2 | 180 | |
| | (equivalent to | | | | |
| | sodium hyaluronate | | | | |
| | of 0.05) | | | | |
| G4 | Sodium hyaluronate | Glycerin/ | Butylene glycol/ | make up to | 6.1 ± 1.37 |
| | solution/12 | 30 | 2 | 180 | |
| | (equivalent to | | | | |
| | sodium hyaluronate | | | | |
| | of 0.12) | | | | |
| G5 | Sodium hyaluronate | Glycerin/ | Butylene glycol/ | make up to | 4.7 ± 1.55 |
| | solution/12 | 5 | 2 | 180 | |
| | (equivalent to | | | | |
| | sodium hyaluronate | | | | |
| | of 0.12) | | | | |
| H1 | Sodium hyaluronate | Glycerin/ | Butylene glycol/ | make up to | 14.3 ± 2.77 |
| | solution/16 | 14 | 1 | 180 | |
| | (equivalent to | | | | |
| | sodium hyaluronate | | | | |
| | of 0.16) | | | | |
| H2 | Sodium hyaluronate | Glycerin/ | Allantoin/ | make up to | 10.5 ± 2.87 |
| | solution/16 | 14 | 1 | 180 | |
| | (equivalent to | | | | |
| | sodium hyaluronate | | | | |
| | of 0.16) | | | | |
| H3 | Sodium hyaluronate | Glycerin/ | Propylene glycol/ | make up to | 10.32 ± 2.01 |
| | solution/16 | 14 | 1 | 180 | |
| | (equivalent to | | | | |
| | sodium hyaluronate | | | | |
| | of 0.16) | | | | |
| H4 | Sodium hyaluronate | Glycerin/ | Butylene glycol/ | make up to | 14.5 ± 2.17 |
| | solution/16 | 14 | 3 | 180 | |
| | (equivalent to | | | | |
| | sodium hyaluronate | | | | |
| | of 0.16) | | | | |
| H5 | Sodium hyaluronate | Glycerin/ | Allantoin/ | make up to | 11.3 ± 2.43 |
| | solution/16 | 14 | 3 | 180 | |
| | (equivalent to | | | | |
| | sodium hyaluronate | | | | |
| | of 0.16) | | | | |
| H6 | Sodium hyaluronate | Glycerin/ | Propylene glycol/ | make up to | 10.87 ± 2.34 |
| | solution/16 | 14 | 3 | 180 | |
| | (equivalent to | | | | |
| | sodium hyaluronate | | | | |
| | of 0.16) | | | | |
| H7 | Sodium hyaluronate | Glycerin/ | Butylene glycol/ | make up to | 13.8 ± 2.76 |
| | solution/15 | 15 | 2 | 180 | |
| | (equivalent to | | | | |
| | sodium hyaluronate | | | | |
| | of 0.15) | | | | |
| H8 | Sodium hyaluronate | Glycerin/ | Allantoin/ | make up to | 10.1 ± 1.99 |
| | solution/15 | 15 | 2 | 180 | |
| | (equivalent to | | | | |
| | sodium hyaluronate | | | | |
| | of 0.15) | | | | |
| H9 | Sodium hyaluronate | Glycerin/ | Propylene | make up to | 9.98 ± 1.98 |
| | solution/15 | 15 | glycol/2 | 180 | |
| | (equivalent to | | | | |
| | sodium hyaluronate | | | | |
| | of 0.15) | | | | |
| H10 | Sodium hyaluronate | Glycerin/ | Butylene glycol/ | make up to | 13.1 ± 1.77 |
| | solution/14 | 16 | 2 | 180 | |
| | (equivalent to | | | | |
| | sodium hyaluronate | | | | |
| | of 0.14) | | | | |
| H11 | Sodium hyaluronate | Glycerin/ | Allantoin/ | make up to | 9.6 ± 2.03 |
| | solution/14 | 16 | 2 | 180 | |
| | (equivalent to | | | | |
| | sodium hyaluronate | | | | |
| | of 0.14) | | | | |
| H12 | Sodium hyaluronate | Glycerin/ | Propylene glycol/ | make up to | 8.97 ± 2.09 |
| | solution/14 | 16 | 2 | 180 | |
| | (equivalent to | | | | |
| | sodium hyaluronate | | | | |
| | of 0.14) | | | | |
| H13 | Sodium hyaluronate | Glycerin/ | Butylene glycol/ | make up to | 12.1 ± 1.99 |
| | solution/13 | 17 | 2 | 180 | |
| | (equivalent to | | | | |
| | sodium hyaluronate | | | | |
| | of 0.13) | | | | |
| H14 | Sodium hyaluronate | Glycerin/ | Allantoin/ | make up to | 8.7 ± 2.03 |
| | solution/13 | 17 | 2 | 180 | |
| | (equivalent to | | | | |
| | sodium hyaluronate | | | | |
| | of 0.13) | | | | |
| H15 | Sodium hyaluronate | Glycerin/ | Propylene glycol/ | make up to | 8.32 ± 2.33 |
| | solution/13 | 17 | 2 | 180 | |
| | (equivalent to | | | | |
| | sodium hyaluronate | | | | |
| | of 0.13) | | | | |
| H16 | Sodium hyaluronate | Glycerin/18 | Butylene glycol/ | make up to | 11.7 ± 2.33 |
| | solution/12 | | 2 | 180 | |
| | (equivalent to | | | | |
| | sodium hyaluronate | | | | |
| | of 0.12) | | | | |
| H17 | Sodium hyaluronate | Glycerin/ | Allantoin/2 | make up to | 7.2 ± 2.98 |
| | solution/12 | 18 | | 180 | |
| | (equivalent to | | | | |
| | sodium hyaluronate | | | | |
| | of 0.12) | | | | |
| H18 | Sodium hyaluronate | Glycerin/ | Propylene glycol/ | make up to | 7.98 ± 2.56 |
| | solution/12 | 18 | 2 | 180 | |
| | (equivalent to | | | | |
| | sodium hyaluronate | | | | |
| | of 0.12) | | | | |

Note: The sodium hyaluronate solution in component A in Table 3 was an aqueous solution with a mass fraction of sodium hyaluronate of 100.

The glove prepared by applying the coating was examined for the change in skin hydration on the back of the hands of the wearer after a period of wearing. The results showed that the glove comprising the coating of each group increased the skin hydration of the wearer after removing the gloves, and no redness was found in any of the experiment subjects.

It was found that, as the dosage of glove coating formulation, among the groups G1-G5, the group G1 had the best effect, which was significantly better than group G2 (P<0.05), group G3 (P<0.05), group G4 (P<0.05), or group G5 (P<0.05), which can be seen that the regulation of the suitable ratio of sodium hyaluronate and glycerol, within a certain range of the dosage, can better improve the wearer's skin hydration.

Also, as the formulation dosage of the glove coating, the group H1-H18 had comparatively good effect. Therefore, if sodium hyaluronate+glycerin, butylene glycol, allantoin or propylene glycol were adjusted to an appropriate ratio, and in a certain range of dosage, the skin hydration of the wearer can be better improved.

### Example 7 Mechanical Property Test of Gloves

The gloves comprising the coating prepared in group B8, group C10, group D13, group E16 and group F19 of Example 4 were tested for glove performance in accordance with U.S. Standard ASTM D6319 and the results were shown in Table 4.

TABLE 4
Mechanical property test results of gloves.

| | Maximum | Tensile | Elongation |
|---|---|---|---|
| Group | force/N | strength/Mpa | at break/% |
| B8 | 11.040 | 28.643 | 597.006 |
| C10 | 10.279 | 26.919 | 541.604 |
| D13 | 9.922 | 24.915 | 515.469 |
| E16 | 10.985 | 29.951 | 520.088 |
| F19 | 11.040 | 28.643 | 597.006 |

The results showed that the gloves comprising the coating prepared in group B8, group C10, group D13, group E16, and group F19, had a tensile strength greater than or equal to 25 MPa and an elongation at break greater than or equal to 500%, which had better tensile strength and elongation at break than ordinary pure nitrile gloves.

The above embodiments are only used to illustrate the technical solutions of the present disclosure and not to limit the scope of protection of the present disclosure. Although the present disclosure is described in detail with reference to the better embodiments, it should be understood by the ordinarily skilled in the prior art that the technical solutions of the present disclosure may be modified or equivalent substituted without departing from the substance and scope of the technical solutions of the present disclosure.

What is claimed is:

1. A glove having an inner surface for contact with skin of a subject's hand, wherein the inner surface is provided with a coating comprising hyaluronic acid or its salt thereof, and glycerin.

2. The glove according to claim 1, wherein the coating on the inner surface of the glove increases skin hydration of the subject's hand upon contact therewith.

3. The glove according to claim 1, wherein the coating comprises, by mass parts, 0.06-0.29 parts of hyaluronic acid or its salt thereof, and 6-29 parts of glycerin.

4. The glove according to claim 1, wherein the coating comprises, by mass parts, 0.1-0.2 parts of hyaluronic acid or its salt thereof, and 10-20 parts of glycerin.

5. The glove according to claim 1, wherein the coating comprises, by mass parts, 0.11-0.18 parts of hyaluronic acid or its salt thereof, and 11-18 parts of glycerin.

6. The glove according to claim 1, wherein the coating comprises, by mass parts, 0.12-0.17 parts of hyaluronic acid or its salt thereof, and 12-17 parts of glycerin.

7. The glove according to claim 1, wherein the coating comprises, by mass parts, 0.13-0.17 parts of hyaluronic acid or its salt thereof, and 13-17 parts of glycerin.

8. The glove according to claim 1, wherein the coating comprises, by mass parts, 0.14-0.17 parts of hyaluronic acid or its salt thereof, and 13-16 parts of glycerin.

9. The glove according to claim 1, wherein the coating comprises, by mass parts, 0.14-0.16 parts of hyaluronic acid or its salt thereof, and 14-16 parts of glycerin.

10. The glove according to claim 1, wherein the coating comprises, by mass parts, 0.15-0.16 parts of hyaluronic acid or its salt thereof, and 14-15 parts of glycerin.

11. The glove according to claim 1, wherein the coating comprises, by mass parts, 0.16 parts of hyaluronic acid or its salt thereof, and 14 parts of glycerin.

12. The glove according to claim 1, wherein the coating further comprises butylene glycol, allantoin, or propylene glycol.

13. The glove according to claim 1, wherein the coating comprises, by mass parts, 1-5 parts butylene glycol, allantoin, or propylene glycol.

14. The glove according to claim 1, wherein the coating comprises, by mass parts, 3-4 parts butylene glycol, allantoin, or propylene glycol.

15. The glove according to claim 1, wherein the coating comprises, by mass parts, 2 parts butylene glycol, allantoin, or propylene glycol.

16. The glove according to claim 1, wherein the salt is sodium hyaluronate, potassium hyaluronate, or calcium hyaluronate.

17. The glove according to claim 1, wherein the glove is a nitrile glove.

18. A preparation method of a glove, comprising:
providing a glove having an inner surface for contact with skin of a subject's hand, wherein the inner surface is provided with a coating comprising hyaluronic acid or its salt thereof, and glycerin.

19. The preparation method according to claim 18, wherein the coating comprises, by mass parts, 0.06-0.29 parts of hyaluronic acid or its salt thereof, and 6-29 parts of glycerin.

20. The preparation method according to claim 18, wherein the coating further comprises one of butylene glycol, allantoin, or propylene glycol.
